(19) United States
(12) Patent Application Publication
(10) Pub. No.: US 2017/0306517 A1
(43) Pub. Date: Oct. 26, 2017

(54) COMPOSITIONS FOR ELECTRODEPOSITION OF METALS, ELECTRODEPOSITION PROCESS AND PRODUCT OBTAINED

(71) Applicant: SOLVAY SPECIALTY POLYMERS ITALY S.P.A., Bollate (IT)

(72) Inventors: Paula COJOCARU, Legnano (IT); Cristiano MONZANI, Trezzo Sull'adda (IT); Marco APOSTOLO, Senago (IT); Francesco Maria TRIULZI, Garbagnate Milanese (IT); Vito TORTELLI, Milano (IT); Elvira GOMEZ VALENTIN, Barcelona (ES); Elisa VALLES GIMENEZ, Barcelona (ES)

(21) Appl. No.: 15/518,182

(22) PCT Filed: Oct. 8, 2015
(86) PCT No.: PCT/EP2015/073285

§ 371 Date: Apr. 10, 2017

(30) Foreign Application Priority Data
Oct. 10, 2014 (EP) 14382389.6

(51) Int. Cl. C25D 3/66; C07C 311/09; C07C 301/02; C07F 19/00; C07C 211/63

(52) U.S. Cl. CPC C25D 3/66; C07F 19/005; C07C 211/63; C07C 301/02; C07C 311/09

(57) ABSTRACT

The present invention pertains to a composition comprising: (I) at least one ionic liquid of formula (1-a) or of formula (1-b): [RF-CFR′_{F}-SO₃]⁻ A⁺ (1-a) [(RF-CFR′F—SO₂)₂N]⁻ (1-b) wherein: -R_{F} is a C₁-C₂₅ fluoroalkyi group, optionally comprising one or more than one catenary ethereal oxygen atoms, -R′_{F} is —F or a —CF₃ group, and -A+ is an organic cation selected from the group consisting of tetraalkylammonium, pyridinium, imidazolium, piperidinium, pyrrolidinium, amidinium and guanidinium groups, and (II) at least one metal salt of formula (II): MeB, (H) wherein: -Me^{m+} is a metal cation deriving from a metal (Me) selected from the group consisting of groups IB, MB, IVB, VB, VIB, MIA, IVA and VIII (8, 9, 10) of the Periodic Table, preferably from the group consisting of groups IVB, VB, VIB and IMA of the Periodic Table, wherein m is the valence of said metal cation, and —Bⁿ⁻ is an inorganic anion, wherein n is the valence of said inorganic anion. The present invention also pertains to the use of said composition in an electrodeposition process and to the metal-coated assembly thereby provided.

This application claims priority to European application No. EP 14382389.6 filed on Oct. 10, 2014, the whole content of this application being incorporated herein by reference for all purposes.

### TECHNICAL FIELD

The present invention pertains to a composition comprising a metal salt suitable for use in an electrodeposition process, to said electrodeposition process and to the metal-coated assembly thereby provided.

### BACKGROUND ART

Electrodeposition is a commonly known technique for depositing a metal coating onto a conductive substrate.

Most commonly employed substrates include those made from metals such as iron, steel, copper, zinc, brass, tin, nickel, chromium and aluminium, as well as pre-treated metals.

Thorough surface cleaning and activation of such metal substrates is typically required to ensure adequate adhesion and coverage of the coatings electrodeposited onto the substrate.

Water is widely used as liquid medium in the electrodeposition of a metal coating onto a conductive substrate. However, only metals having reduction potential higher than that of hydrogen can be electrodeposited from aqueous solutions.

On the other side, metals having negative reduction potential such as aluminium, titanium, zirconium, hafnium, vanadium, niobium, tantalum, molybdenum and tungsten cannot be electrodeposited from aqueous solutions due to a massive hydrogen evolution at the cathode. Hence, solutions of electrolytes in organic aprotic solvents or ionic liquids, preferably free from moisture, must be used for the electrodeposition of these metals.

Solutions of metal salts such as metal halide salts in organic aprotic solvents may be applied in the process for the electrodeposition of metals having negative reduction potential. However, the electrodeposition of these metals from solutions in organic aprotic solvents has limited applicability due to the narrow electrochemical stability window, low electrical conductivity, high volatility and high flammability of these solvents.

Salts having a low melting point which are liquid at room temperature, or even below, which form a class of liquids usually called ionic liquids, have also been used in the process for the electrodeposition of metals having negative reduction potential. In particular, ionic liquids consisting of 1,3-dialkylimidazolium or 1,1-dialkylpyrrolidinium cations and anions such as trifluoromethylsulphonate or bis(trifluoromethylsulphonyl)imide have been investigated. However, these ionic liquids are typically air and moisture sensitive.

Moreover, non-uniform electrodeposited metal coatings are usually obtained by using traditional electrodeposition processes which suffer from poor adhesion to the substrate.

Further, most metals and metal alloys naturally form an oxide layer upon exposure to air. This oxide layer generally forms a physical barrier to the metal coatings and must be removed or prevented for forming.

There is thus still the need in the art for compositions suitable for use in processes for the electrodeposition of metal coatings onto a substrate which enable obtaining homogeneous metal coatings having a uniform thickness and good adhesion to the substrate.

### SUMMARY OF INVENTION

It has been now found that by using certain ionic liquids it is possible to obtain an air and moisture stable composition suitable for use in a process for the electrodeposition of a metal layer onto a conductive substrate.

In particular, the composition of the present invention is suitable for use in a process for the electrodeposition of a metal layer onto a conductive substrate under air atmosphere.

The composition of the present invention unexpectedly exhibits a good electrochemical stability in a wide electrochemical window. Also, the composition of the present invention advantageously enables solubilising one or more metal salts in a wide range of concentrations in a wide range of temperatures.

The process of the invention successfully enables obtaining a homogeneous metal layer, typically formed of a plurality of nano-aggregates, which advantageously uniformly covers the surface of the conductive substrate. The metal layer obtainable by the process of the invention also advantageously has a uniform thickness and is well adhered to the conductive substrate.

In a first instance, the present invention pertains to a composition comprising:

(I) at least one ionic liquid of formula (I-a) or of formula (I-b):

[R_{F}-CFR′_{F}-SO₃]⁻ A⁺ (I-a)

[(R_{F}-CFR′_{F}-SO₂)₂N]⁻ A⁺ (I-b)

wherein:
R_{F} is a C₁-C₂₅ fluoroalkyl group, optionally comprising one or more than one catenary ethereal oxygen atoms,
R′_{F} is —F or a —CF₃ group, and
A⁺ is an organic cation selected from the group consisting of tetraalkylammonium, pyridinium, imidazolium, piperidinium, pyrrolidinium, amidinium and guanidinium groups, and

(II) at least one metal salt of formula (II):

MeₙBₘ (II)

wherein:
Me^{m+} is a metal cation deriving from a metal (Me) selected from the group consisting of groups IB, IIB, IVB, VB, VIB, IIIA, IVA and VIII (8, 9, 10) of the Periodic Table, preferably from the group consisting of groups IVB, VB, VIB and IIIA of the Periodic Table, wherein m is the valence of said metal cation, and
Bⁿ⁻ is an inorganic anion, wherein n is the valence of said inorganic anion.

The composition of the invention is advantageously in the form of a solution.

For the purpose of the present invention, the term “solution” is intended to denote a uniformly dispersed mixture of at least one metal salt of formula (II), typically referred to as solute, in at least one ionic liquid of formula (I-a) or of formula (I-b), typically referred to as solvent. The term “solvent” is used herein in its usual meaning, that is to say that it refers to a substance capable of dissolving a solute. It is common practice to refer to a solution when the resulting mixture is clear and no phase separation is visible in the system. Phase separation is taken to be the point, often referred to as “cloud point”, at which the solution becomes turbid or cloudy due to the formation of polymer aggregates or at which the solution turns into a gel.

The Applicant thinks, without this limiting the scope of the invention, that ionic liquids of formula (I-a) or of formula (I-b) comprising a specific fluoroalkyl group R_{F} advantageously exhibit good electrochemical stability in a wide electrochemical window and advantageously provide compositions enabling solubilising one or more metal salts in a wide range of concentrations in a wide range of temperatures, while successfully being air and moisture stable to be suitable for use in an electrodeposition process.

In a second instance, the present invention pertains to an electrodeposition process comprising:

(i) providing an electrolytic cell comprising:
a conductive substrate, and
a positive electrode,

said conductive substrate and said positive electrode being immersed in a composition comprising:

(I) at least one ionic liquid of formula (I-a) or of formula (I-b):

[R_{F}-CFR′_{F}-SO₃]⁻ A⁺ (I-a)

[(R_{F}-CFR_{F}-SO₂)₂N]⁻ A⁺ (I-b)

wherein:
R_{F} is a C₁-C₂₅ fluoroalkyl group, optionally comprising one or more than one catenary ethereal oxygen atoms,
R′_{F} is —F or a —CF₃ group, and
A⁺ is an organic cation selected from the group consisting of tetraalkylammonium, pyridinium, imidazolium, piperidinium, pyrrolidinium, amidinium and guanidinium groups, and

(II) at least one metal salt of formula (II):

MeₙBₘ (II)

wherein:
Me^{m+} is a metal cation deriving from a metal (Me) selected from the group consisting of groups IB, IIB, IVB, VB, VIB, IIIA, IVA and VIII (8, 9, 10) of the Periodic Table, preferably from the group consisting of groups IVB, VB, VIB and IIIA of the Periodic Table, wherein m is the valence of said metal cation, and
Bⁿ⁻ is an inorganic anion, wherein n is the valence of said inorganic anion; and

(ii) driving an electric current through the electrolytic cell provided in step (i).

The composition of the invention is particularly suitable for use in the electrodeposition process of the invention.

For the purpose of the present invention, the term “conductive” is intended to denote a substrate having an electrical resistivity of at most 50 Ω/square, preferably of at most 25 Ω/square, more preferably of at most 20 Ω/square, even more preferably of at most 15 Ω/square.

Under the electrodeposition process of the invention, the conductive substrate typically operates as a negative electrode.

For the purpose of the present invention, the term “electrodeposition” is intended to denote a process carried out in an electrolytic cell wherein electrons flow through an electrolytic composition from a positive electrode to a negative electrode thereby causing an inorganic anion (Bⁿ⁻) in the composition to be oxidised at the positive electrode and a metal cation (Me^{m+}) in the composition to be reduced at the negative electrode so that a layer made of a metal in its elemental state (Me) is deposited onto said negative electrode.

For the purpose of the present invention, the term “positive electrode” is intended to denote the anode where oxidation takes place. For the purpose of the present invention, the term “negative electrode” is intended to denote the cathode where reduction takes place.

Under step (i) of the electrodeposition process of the invention, the electrolytic cell typically further comprises a counter electrode.

For the purpose of the present invention, the term “counter electrode” is intended to denote the electrode through which the electric current that flows via the negative electrode into the electrolytic composition leaves the composition.

The electrodeposition process of the invention may be carried out either under inert atmosphere or under air atmosphere.

The electrodeposition process of the invention is advantageously carried out under air atmosphere.

The electrodeposition process of the invention is typically carried out at a temperature of at most 120° C. The electrodeposition process of the invention is typically carried out at a temperature of at least 20° C.

The electrodeposition process of the invention advantageously enables obtaining a metal-coated assembly comprising:
a conductive substrate, and
adhered to at least a portion of at least one surface of said conductive substrate, a layer made of a metal (Me) selected from the group consisting of groups IB, IIB, IVB, VB, VIB, IIIA, IVA and VIII (8, 9, 10) of the Periodic Table, preferably from the group consisting of groups IVB, VB, VIB and IIIA of the Periodic Table.

Thus, in a third instance, the present invention pertains to a metal-coated assembly obtainable by the electrodeposition process of the invention, said metal-coated assembly comprising:
a conductive substrate, and
adhered to at least a portion of at least one surface of said conductive substrate, a layer made of a metal (Me) selected from the group consisting of groups IB, IIB, IVB, VB, VIB, IIIA, IVA and VIII (8, 9, 10) of the Periodic Table, preferably from the group consisting of groups IVB, VB, VIB and IIIA of the Periodic Table.

The conductive substrate of the metal-coated assembly of the invention is typically the negative electrode of the electrolytic cell of the electrodeposition process of the invention.

The composition of the invention typically comprises:

(I) from 20% to 95% by weight, based on the total weight of the composition, of at least one ionic liquid of formula (I-a) or of formula (I-b):

[R_{F}-CFR′_{F}-SO₃]⁻ A⁺ (I-a)

[(R_{F}-CFR_{F}-SO₂)₂N]⁻ A⁺ (I-b)

wherein:
R_{F} is a C₁-C₂₅ fluoroalkyl group, optionally comprising one or more than one catenary ethereal oxygen atoms,
R′_{F} is —F or a —CF₃ group, and
A⁺ is an organic cation selected from the group consisting of tetraalkylammonium, pyridinium, imidazolium, piperidinium, pyrrolidinium, amidinium and guanidinium groups, and

(II) from 5% to 80% by weight, based on the total weight of the composition, of at least one metal salt of formula (II):

MeₙBₘ (II)

wherein:
Me^{m+} is a metal cation deriving from a metal (Me) selected from the group consisting of groups IB, IIB, IVB, VB, VIB, IIIA, IVA and VIII (8, 9, 10) of the Periodic Table, preferably from the group consisting of groups IVB, VB, VIB and IIIA of the Periodic Table, wherein m is the valence of said metal cation, and
Bⁿ⁻ is an inorganic anion, wherein n is the valence of said inorganic anion.

The ionic liquid of formula (I-a) or of formula (I-b) advantageously has a melting point of at most 120° C., preferably of at most 100° C., more preferably of at most 90° C.

The ionic liquid of formula (I-a) or of formula (I-b) is typically liquid at temperatures below 120° C. under atmospheric pressure.

The ionic liquid of formula (I-a) or of formula (I-b) is thus particularly suitable for use in the process of the invention.

For the purpose of the present invention, the term “fluoroalkyl” is intended to denote either a per(halo)fluorinated alkyl group, wherein all the hydrogen atoms of the alkyl group are replaced by fluorine atoms and, optionally, one or more than one halogen atoms different from fluorine atoms, or a partially fluorinated alkyl group, wherein only a part of the hydrogen atoms of the alkyl group are replaced by fluorine atoms and, optionally, one or more than one halogen atoms different from fluorine atoms.

The fluoroalkyl group R_{F} is typically selected from the group consisting of:
a C₁-C₂₅ per(chloro)fluorinated alkyl group, optionally comprising one or more than one catenary ethereal oxygen atoms, wherein all the hydrogen atoms of the alkyl group are replaced by fluorine atoms and, optionally, one or more than one chlorine atoms, and
a C₁-C₂₅ partially fluorinated alkyl group, optionally comprising one or more than one catenary ethereal oxygen atoms, wherein only a part of the hydrogen atoms of the alkyl group are replaced by fluorine atoms and, optionally, one or more than one chlorine atoms.

The fluoroalkyl group R_{F} is preferably a C₁-C₁₀ fluoroalkyl group, more preferably a C₁-C₆ fluoroalkyl group, even more preferably a C₂-C₄ fluoroalkyl group, optionally comprising one or more than one catenary ethereal oxygen atoms.

The ionic liquid preferably has formula (I′-a) or formula (I′-b):

[R_{F1}-CF₂—SO₃]⁻ A⁺ (I′-a)

[(R_{F1}-CF₂—SO₂)₂N]⁻ A⁺ (I′-b)

wherein:
R_{F1} is selected from the group consisting of —CF₃, —CF₂H, —CFHCl, —CF₂ CF₃, —CFHCF₃, —CFHOCF₃, —CF₂CF₂CF₃, —CF₂OCF₂CF₃, —CFHOCF₂CF₃, —CF₂OCFHOF₃, —CF₂OCF₂CF₂H, —CF₂OCF₂CF₂C1, —CF₂OCFClCF₂Cl, —CFHOCF₂CF₂CF₃, —CF₂OCF₂CF₂OCF₂CF₂CF₃ and —CF₂OCF(CF₃)OCF₂CF₂CF₃, and
A⁺ is an organic cation selected from the group consisting of tetraalkylammonium, pyridinium, imidazolium, piperidinium, pyrrolidinium, amidinium and guanidinium groups.

The tetraalkylammonium group typically has formula (I-A):

[NR¹R²R³R⁴] (I-A)

wherein R¹, R², R³ and R⁴, equal to or different from each other, are independently selected from the group consisting of C₁-C₂₅, preferably C₂-C₂₀, straight-chain, branched or cyclic, optionally substituted, alkanes or alkenes, and C₆-C₂₅, optionally substituted, aryl or heteroaryl groups.

Preferably, in formula (I-A), R¹, R², R³ and R⁴, equal to or different from each other, are independently selected from the group consisting of C₁-C₁₀ straight-chain, branched or cyclic alkanes.

The pyridinium group typically has formula (I-B):

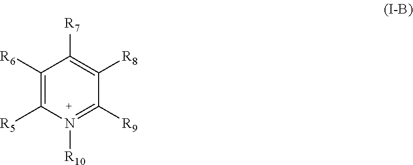

wherein R′⁵, R⁶, R⁷, R⁸, R⁹ and R¹⁰, equal to or different from each other, are independently selected from the group consisting of hydrogen atoms, halogen atoms, C₁-C₂₅, preferably C₁-C₂₀, straight-chain, branched or cyclic, optionally substituted, alkanes or alkenes, and C₆-C₂₅, optionally substituted, aryl or heteroaryl groups.

Preferably, in formula (I-B), R′⁵, R⁶, R⁷, R⁸, R⁹ and R¹⁹, equal to or different from each other, are independently selected from the group consisting of hydrogen atoms and C₁-C₂₅, preferably C₁-C₂₀, straight-chain, branched or cyclic, optionally substituted, alkanes.

Non-limiting examples of suitable pyridinium groups of formula (I-B) are for instance those having:
formula (I-B1) wherein R′⁵, R⁶, R⁷, R⁸, R⁹ and R¹⁰ are hydrogen atom or
formula (I-B2) wherein R′⁵=R⁹=R¹⁰=—CH₃ and R⁶=R⁷=R⁸=H or
formula (I-B3) wherein R′⁵=R⁷=R⁹=R¹⁰=—CH₃ and R⁶=R⁸=H or
formula (I-B4) wherein R′⁵=R⁷=R¹⁰=—CH₃ and R⁶=R⁸=R⁹=H.

The amidinium group typically has formula (I-C):

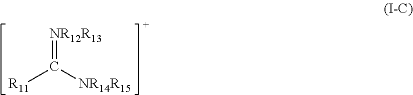

wherein R¹¹, R¹², R¹³, R¹⁴ and R¹⁵, equal to or different from each other, are independently selected from the group consisting of hydrogen atoms and C₁-C₂₅, preferably C₁-C₂₀, straight-chain, branched or cyclic, optionally substituted, alkanes or alkenes, optionally comprising heteroatoms.

Preferably, in formula (I-C), R¹¹, R¹², R¹³, R¹⁴ and R¹⁵ are not all simultaneously hydrogen atoms.

In formula (I-C), R¹¹, R¹², R¹³, R¹⁴ and R¹⁵ may be bonded in pairs in such a way that mono-, bi- or poly-cyclic amidinium groups are provided.

Non-limiting examples of preferred amidinium groups of formula (I-C) are those derived from 1,8-diazabicyclo[5.4.0]undec-7-ene, 1,5-diazabicyclo[4.3.0]non-5-ene, 2,9-diazabicyclo[4.3.0]non-1,3,5,7-tetraene and 6-(dibutylamino)-1,8-diazabicyclo[5.4.0]undecene-7.

The guanidinium group typically has formula (I-D):

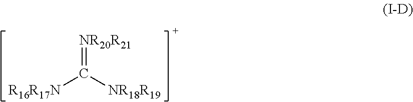

wherein R¹⁶, R¹⁷, R¹⁸, R¹⁹, R²⁰ and R²¹, equal to or different from each other, are independently selected from the group consisting of hydrogen atoms and C₁-C₂₅, preferably C₁-C₂₀, straight-chain, branched or cyclic, optionally substituted, alkanes or alkenes, optionally comprising heteroatoms.

Preferably, in formula (I-D), R¹⁶, R¹⁷, R¹⁸, R¹⁹, R²⁰ and R²¹ are not simultaneously hydrogen atoms, more preferably at least one of R¹⁶ R¹⁷, R¹⁸, R¹⁹, R²⁰ and R²¹ is a hydrogen atom.

In formula (I-D), R¹⁶, R¹⁷, R¹⁸, R¹⁹, R²⁰ and R²¹ may be bonded in pairs in such a way that mono-, bi- or poly-cyclic guanidinium groups are provided.

Non-limiting examples of preferred guanidinium groups of formula (I-D) are those derived from 1-methylguanidine, 1-ethylguanidine, 1-cyclohexylguanidine, 1-phenylguanidine, 1,1-dimethylguanidine, 1,3-dimethylguanidine, 1,2-diphenylguanidine, 1,1,2-trimethylguanidine, 1,2,3-tricyclohexylguanidine, 1,1,2,2-tetramethylguanidine, guanine, 1,5,7-triazabicyclo[4.4.0]-dec-5-ene, 7-methyl-1,5,7-triazabicyclo[4.4.0]dec-5-ene, 7-ethyl-1,5,7-triazabicyclo[4.4.0]dec-5-ene, 7-n-propyl-1,5,7-triazabicyclo[4.4.0]dec-5-ene, 7-isopropyl-1,5,7-triazabicyclo[4.4.0]dec-5-ene, 7-n-butyl-1,5,7-triazabicyclo[4.4.0]dec-5-ene, 7-cyclohexyl-1,5,7-triazabicyclo[4.4.0]dec-5-ene and 7-n-octyl-1,5,7-triazabicyclo[4.4.0]dec-5-ene.

The ionic liquid of formula (I-a) or of formula (I-b) is typically obtainable by a process comprising reacting a fluoroalkyl sulfonyl halide with an organic base selected from the group consisting of tetraalkylamines, pyridines, amidines and guanidines.

The fluoroalkyl sulfonyl halide is typically of formula (I-a1) or of formula (I-b1):

[R_{F}-CFR′_{F}-SO₃]⁻ A⁺ (I-a)

[(R_{F}-CFR′_{F}-SO₂)₂N]⁻ A⁺ (I-b)

wherein:
R_{F} is a C₁-C₂₅ fluoroalkyl group, optionally comprising one or more than one catenary ethereal oxygen atoms,
R′_{F} is —F or a —CF₃ group, and
X is selected from the group consisting of F, Cl and Br, preferably from the group consisting of F and Cl, more preferably X is F.

Tetraalkylamines suitable for use in the process of the invention are typically selected from the group consisting of those of formula (I-2A):

NR′¹R′²R′³ (I-2A)

wherein R′¹, R′² and R′³, equal to or different from each other, are independently selected from the group consisting of C₁-C₂₅, preferably C₂-C₂₀, straight-chain, branched or cyclic, optionally substituted, alkanes or alkenes, and C₆-C₂₅, optionally substituted, aryl or heteroaryl groups.

Preferably, in formula (I-2A), R′¹, R′² and R′³, equal to or different from each other, are independently selected from the group consisting of C₁-C₁₀ straight-chain, branched or cyclic alkanes.

Pyridines suitable for use in the process of the invention are typically selected from the group consisting of those of formula (I-2B):

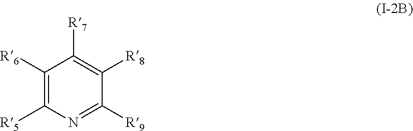

wherein R′⁵, R′⁶, R′⁷, R′⁸ and R′⁹, equal to or different from each other, are independently selected from the group consisting of hydrogen atoms, halogen atoms, C₁-C₂₅, preferably C₁-C₂₀, straight-chain, branched or cyclic, optionally substituted, alkanes or alkenes, and C₆-C₂₅, optionally substituted, aryl or heteroaryl groups.

Preferably, in formula (I-2B), R′⁵, R′⁶, R′⁷, R′⁸ and R′⁹, equal to or different from each other, are independently selected from the group consisting of hydrogen atoms and C₁-C₂₅, preferably C₁-C₂₀, straight-chain, branched or cyclic, optionally substituted, alkanes.

Non-limiting examples of suitable pyridines of formula (I-2B) are for instance those having:
formula (I-2B1) wherein R′⁵, R′⁶, R′⁷, R′⁸ and R′⁹ are hydrogen atom or
formula (I-2B2) wherein R′⁵=R′⁹=—CH₃ and R′⁶=R′⁷=R′⁸=H or
formula (I-2B3) wherein R′⁵=R′⁷=R′⁹=—CH₃ and R′⁶=R′⁸=H or
formula (I-2B4) wherein R′⁵=R′⁷=—CH₃ and R′⁶=R′⁸=R′⁹=H.

Amidines suitable for use in the process of the invention are typically selected from the group consisting of those of formula (I-2C):

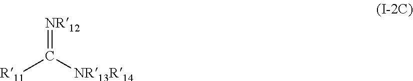

wherein R′¹¹, R′¹², R′¹³ and R′¹⁴, equal to or different from each other, are independently selected from the group consisting of hydrogen atoms and C₁-C₂₅, preferably C₁-C₂₀, straight-chain, branched or cyclic, optionally substituted, alkanes or alkenes, optionally comprising heteroatoms.

Preferably, in formula (I-2C), R′¹¹, R′¹², R′¹³ and R′¹⁴ are not all simultaneously hydrogen atoms.

In formula (I-2C), R′¹¹, R′¹², R′¹³ and R¹⁴ may be bonded in pairs in such a way that mono-, bi- or poly-cyclic amidines are provided.

Non-limiting examples of preferred amidines of formula (I-2C) include, notably, 1,8-diazabicyclo[5.4.0]undec-7-ene, 1,5-diazabicyclo[4.3.0]non-5-ene, 2,9-diazabicyclo[4.3.0]non-1,3,5,7-tetraene and 6-(dibutylamino)-1,8-diazabicyclo[5.4.0]undecene-7.

Guanidines suitable for use in the process of the invention are typically selected from the group consisting of those of formula (I-2D):

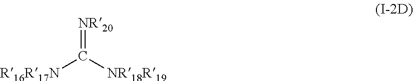

wherein R′¹⁶, R′¹⁷, R′¹⁸, R′¹⁹ and R′²⁰, equal to or different from each other, are independently selected from the group consisting of hydrogen atoms and C₁-C₂₅, preferably C₁-C₂₀, straight-chain, branched or cyclic, optionally substituted, alkanes or alkenes, optionally comprising heteroatoms.

Preferably, in formula (I-2D), R′¹⁶, R′¹⁷, R′¹⁸, R′¹⁹ and R′²⁰ are not simultaneously hydrogen atoms, more preferably at least one of R′¹⁶, R′¹⁷, R′¹⁸, R′¹⁹ and R′²⁰ is a hydrogen atom.

In formula (I-2D), R′¹⁶, R′¹⁷, R′¹⁸, R′¹⁹ and R′²⁰ may be bonded in pairs in such a way that mono-, bi- or poly-cyclic guanidines are provided.

Non-limiting examples of preferred guanidines of formula (I-2D) include, notably, 1-methylguanidine, 1-ethylguanidine, 1-cyclohexylguanidine, 1-phenylguanidine, 1,1-dimethylguanidine, 1,3-dimethylguanidine, 1,2-diphenylguanidine, 1,1,2-trimethylguanidine, 1,2,3-tricyclohexylguanidine, 1,1,2,2-tetramethylguanidine, guanine, 1,5,7-triazabicyclo[4.4.0]-dec-5-ene, 7-methyl-1,5,7-triazabicyclo[4.4.0]dec-5-ene, 7-ethyl-1,5,7-triazabicyclo[4.4.0]dec-5-ene, 7-n-propyl-1,5,7-triazabicyclo[4.4.0]dec-5-ene, 7-isopropyl-1,5,7-triazabicyclo[4.4.0]dec-5-ene, 7-n-butyl-1,5,7-triazabicyclo[4.4.0]dec-5-ene, 7-cyclohexyl-1,5,7-triazabicyclo[4.4.0]dec-5-ene and 7-n-octyl-1,5,7-triazabicyclo[4.4.0]dec-5-ene.

The organic cation A⁺ of the ionic liquid of formula (I-a) or of formula (I-b) is preferably selected from the group consisting of pyridinium and guanidinium groups.

The ionic liquid more preferably has formula (I″-a) or formula (I″-b):

[R_{F2}-CF₂—SO₃]⁻ A′⁺ (I″-a)

[(R_{F2}-CF₂—SO₂)₂N]⁻ A′⁺ (I″-b)

wherein:
R_{F2} is —CF₂OCFClCF₂Cl or —CF₂OCF₂CF₃, and
A′⁺ is an organic cation selected from the group consisting of pyridinium and guanidinium groups.

The ionic liquid even more preferably has formula (I′″-a) or formula (I′″-b):

[R_{F3}-CF₂—SO₃]⁻ A′⁺ (I′″-a)

[(R_{F3}-CF₂—SO₂)₂N]⁻ A′⁺ (I′″-b)

wherein:
R_{F3} is —CF₂OCFClCF₂Cl, and
A′⁺ is an organic cation selected from the group consisting of pyridinium and guanidinium groups.

For the purpose of the present invention, the term “inorganic” is used according to its usual meaning and is intended to denote an inorganic element or compound which does not contain carbon atoms and is thus not considered an organic element or compound.

The metal salt of formula (II) preferably has formula (II′):

Me′ₙB′ₘ (II′)

wherein:
Me′^{m+} is a metal cation deriving from a metal (Me) selected from the group consisting of groups IB, IIB, IVB, VB, VIB, IIIA, IVA and VIII (8, 9, 10) of the Periodic Table, preferably from the group consisting of groups IVB, VB, VIB and IIIA of the Periodic Table, wherein m is the valence of said metal cation, and
B′ⁿ⁻ is an inorganic anion selected from the group consisting of halides, such as —Cr, —F⁻, —I⁻, —Br, oxohalides, nitrates (—NO₃⁻), sulphates (—SO₄²⁻), sulphites (—SO₃²⁻), sulphamates (—NH₂SO₃₋), oxides (—O²⁻), hydroxides (—OH⁻), phosphates (—PO₄³⁻) and phosphites (−PO₃³⁻).

The metal salt of formula (II) more preferably has formula (II″):

Me″ₙB″ₘ (II″)

wherein:
Me″^{m+} is a metal cation deriving from a metal (Me) selected from the group consisting of aluminium (Al), titanium (Ti), zirconium (Zr), hafnium (Hf), vanadium (V), niobium (Nb), tantalum (Ta), molybdenum (Mo) and tungsten (W), wherein m is the valence of said metal cation, and
B″ⁿ⁻ is an inorganic anion selected from the group consisting of halides, such as —Cr, —F⁻, —I⁻, —Br⁻, oxohalides, nitrates (—NO₃³¹), sulphates (—SO₄²⁻), sulphites (—SO₃²⁻), sulphamates (—NH₂SO₃⁻), oxides (—O²⁻), hydroxides (—OH⁻), phosphates (—PO₄³⁻) and phosphites (—PO₃³⁻).

According to a first embodiment of the invention, the conductive substrate is typically made of a metal selected from the group consisting of groups IB, IIB, IVB, VB, VIB, IIIA, IVA and VIII (8, 9, 10) of the Periodic Table, preferably of a metal selected from the group consisting of iron (Fe), copper (Cu), nickel (Ni), chromium (Cr), manganese (Mn), molybdenum (Mo), titanium (Ti), tin (Sn), zinc (Zn), palladium (Pd), platinum (Pt), silver (Ag), iridium (Ir), indium (In), lead (Pb), tungsten (W), vanadium (V), copper (Cu) and ruthenium (Ru).

According to a second embodiment of the invention, the conductive substrate is typically made of a conductive metal oxide, preferably of a conductive metal oxide selected from the group consisting of ZnO, SnO and tin-doped indium oxide, or of glassy carbon.

The positive electrode is typically made of a metal selected from the group consisting of groups IB, IIB, IVB, VB, VIB, IIIA, IVA and VIII (8, 9, 10) of the Periodic Table, preferably from the group consisting of groups IVB, VB, VIB and IIIA of the Periodic Table.

The positive electrode is more preferably made of aluminium (Al), titanium

(Ti), zirconium (Zr), hafnium (Hf), vanadium (V), niobium (Nb), tantalum (Ta), molybdenum (Mo) and tungsten (W).

The counter electrode, if any, is typically made of platinum (Pt) or graphite.

Should the disclosure of any patents, patent applications, and publications which are incorporated herein by reference conflict with the description of the present application to the extent that it may render a term unclear, the present description shall take precedence.

The invention will be now described in more detail with reference to the following examples whose purpose is merely illustrative and not limitative of the scope of the invention.

Raw Materials

Perfluoro 3-oxa-4,5-dichloro pentyl sulphonate tetramethyl guanidinium salt.

Perfluoro 3-oxa pentyl sulphonate N-methyl-2,4,6-trimethyl pirydinium salt. 1-Ethyl-3-methylimidazolium chloride commercially available from Sigma Aldrich.

1-Ethyl-3-methylimidazolium trifluoromethane sulphonate commercially available from Sigma Aldrich.

Anhydrous AlCl₃ grains commercially available from Sigma Aldrich.

### Example 1

### Perfluoro 3-oxa-4, 5-dichloro pentyl sulphonate tetramethyl guanidinium salt

### Example 1a

### Synthesis of perfluoro 3-oxa-4,5-dichloro pentyl sulphonate tetramethyl guanidinium salt

A three-necked round bottom flask equipped with thermometer, condenser and stirring was charged with 490 ml of a solution of K₂CO₃ in water (4 M), CH₂Cl₂ (490 ml) and N,N,N′,N′-tetramethylguanidine (40 g). CF₂ ClCFClOCF₂CF₂SC₂F (121.90 g) was then added thereto drop-wise. The reaction was stirred at room temperature for 2 hours. A biphasic system was obtained. The organic phase was separated from the aqueous phase, washed with water, treated with MgSO₄ and the solid was filtered off. The product was recovered by evaporation under vacuum in 98% yield (melting point 71° C.; 1% weight loss: 259° C.).

¹⁹F NMR (HFMX reference): −70.9 ppm (d; 2F; ClCF₂₋); −76.5 ppm (m; 1F; —CFClO—); −83.3 ppm (m; 2F; —OCF₂CF₂—); −118.5 ppm (s; 2F; —CF₂SO₃₋).

1H NMR (TMS reference): +2.95 ppm (s; 12H; CH₃N—).

### Example 1b

### Dissolution of AlC13 in perfluoro 3-oxa-4,5-dichloro pentyl sulphonate tetramethyl guanidinium salt

An electrolyte solution was prepared by dissolving AlCl₃ (2 g) in 9 g of perfluoro 3-oxa-4,5-dichloro pentyl sulphonate tetramethyl guanidinium salt. The ionic liquid was melted at 75° C. and AlCl₃ was added thereto under an Argon overflow. Deoxygenation of the solution so obtained was performed with Argon bubbling.

The electrolyte solution so prepared was advantageously clear with no phase separation in a range of temperatures comprised between 20° C. and 120° C.

### Example 2

### Perfluoro 3-oxa pentyl sulphonate N-methyl-2,4,6-trimethyl pirydinium salt

### Example 2a

### Synthesis of perfluoro 3-oxa pentyl sulphonate N-methyl-2,4,6-trimethyl pirydinium salt

A three-necked round bottom flask equipped with thermometer, condenser and stirring was charged with CH₂Cl₂ (80 ml), CH₃OH (4.03 g) and 2,4,6-trimethylpyridine (15.24 g). CF₂ClCFClOCF₂CF₂SO₂F (20 g) was then added thereto drop-wise. The reaction was stirred at room temperature for 2.5 hours. The liquid phase was removed by evaporation under vacuum thereby providing a viscous oil that was re-dissolved in CH₂Cl₂ (200 mL) and extracted with an aqueous 3N NaOH solution (200 mL).

The organic phase was separated from the aqueous phase. The organic phase was treated with Na₂SO₄ and, after filtration, the product was recovered by evaporation under vacuum in 87% yield (melting point 83° C.; 1% weight loss: 323° C.).

¹⁹F NMR (HFMX reference): −84.1 ppm (m; 2F; −OCF₂CF₂—); −88.2 ppm (s; 3F; —CF₃); −90.1 ppm (m; 2F; CF₃CF₂O—); −120.1 ppm (s; 2F; −CF₂SO₃−). 1H NMR (TMS reference): +7.70 ppm (s; 2H; meta-H); +3.96 ppm (s; 3H; NCH₃); +2.72 ppm (s; 6H; ortho-CH₃); +2.47 ppm (s; 3H; para-CH₃).

### Example 2b

### Dissolution of AlC13 in perfluoro 3-oxa pentyl sulphonate N-methyl-2,4,6-trimethyl pirydinium salt

An electrolyte solution was prepared by dissolving AlC1₃ in perfluoro 3-oxa pentyl sulphonate N-methyl-2,4,6-trimethyl pirydinium salt in a 0.4:1 weight ratio. The ionic liquid was melted at 85° C. and AlC1₃ was added thereto under an Argon overflow. Deoxygenation of the solution so obtained was performed with Argon bubbling.

The electrolyte solution so prepared was advantageously clear with no phase separation in a range of temperatures comprised between 20° C. and 120° C.

### Comparative Example 1

### AlC13 in 1-ethyl-3-methylimidazolium chloride

A mixture was prepared by adding AlC1₃ to 1-ethyl-3-methylimidazolium chloride (EMIC) under dry Argon atmosphere inside a glove box. The mixture was prepared by slow addition of AlCl₃ to EMIC under magnetic stirring at room temperature. Attention was paid to avoid thermal degradation of the electrolyte, which can be caused by the highly exothermic reaction between the two components. A 2:1 molar ratio AlCl₃ to EMIC electrolyte mixture was provided.

The electrolyte mixture so prepared was cloudy due to moisture adsorption by the ionic liquid and consequent degradation of the electrolyte thereby contained.

### Comparative Example 2

### AlCl₃ in 1-ethyl-3-methylimidazolium trifluoromethane sulphonate

A 1.6 M solution of AlCl₃ in 1-ethyl-3-methylimidazolium trifluoromethane sulphonate [(EMI)TFO] was prepared inside a glove box containing water and oxygen in an amount below 1 ppm. The mixture was prepared by slow addition of AlCl₃ to (EMI)TFO under magnetic stirring at room temperature. The electrolyte mixture so prepared was cloudy due to moisture adsorption by the ionic liquid and formation of hydrogen bonds between water molecules and [TFO]⁻ anions, as measured by ATR-IR spectroscopy, and consequent degradation of the electrolyte thereby contained.

Electrochemical Measurements

Electrochemical measurements were carried out using a potentiostat either under Argon overflow or upon exposure to air.

Table 1 summarizes the results of cyclic voltammetric experiments recorded on the neat ionic liquids prepared according to Example 1a or Example 2a and on the electrolyte solutions prepared according to Example 1b or Example 2b, using an electrolytic cell comprising Al wire as reference electrode, Pt wire as counter electrode and glassy carbon as working electrode.

The neat ionic liquid prepared according to Example 1a was characterized at 71° C. while the electrolyte solution prepared according to Example 1b was characterized at 75° C. The neat ionic liquid prepared according to Example 2a was characterized at 95° C. while the electrolyte solution prepared according to Example 2b was characterized at 100° C.

Experimental data relative to the electrolyte mixtures prepared according to either Comparative Example 1 or Comparative Example 2 are reported in Table 1 for reference.

TABLE 1

| | | | |
|---|---|---|---|
| | | Al nucleation | |
| | Test temperature | potential | Electrochemical |
| Electrolyte | [° C.] | [V vs Al] | window [V vs Al] |

Test performed in inert environment, dry Argon

| | | | |
|---|---|---|---|
| Ex. 1a | 71 | not applicable | −0.5 to +2.5 |
| Ex. 1b | 75 | −0.5 | not applicable |
| Ex. 2a | 95 | not applicable | −0.8 to +2.5 |
| Ex. 2b | 100 | −0.6 | not applicable |
| C. Ex. 1 | 60 | −0.2 | not applicable |
| C. Ex. 2 | 100 | −1.0 | not applicable |

Test performed in air

| | | | |
|---|---|---|---|
| Ex. 1a | 71 | not applicable | −0.5 to +2.5 |
| Ex. 1b | 75 | −0.5 | not applicable |
| Ex. 2a | 95 | not applicable | −0.8 to +2.5 |
| Ex. 2b | 100 | −0.6 | not applicable |

| | | |
|---|---|---|
| C. Ex. 1 | — | moisture adsorption, electrolyte |
| | | degradation |
| C. Ex. 2 | — | moisture adsorption, electrolyte |
| | | degradation |

Surface Morphology Characterization of the Metal Layer

A homogeneous Al layer of type 1 was observed by SEM images of the surface of the metal layer obtained by electrodeposition onto a glassy carbon electrode according to the process of the invention from an electrolyte solution prepared according to Example 1 b.

The rate numbers reported in Table 2 here below are indicators of the surface properties of a metal layer onto a conductive substrate: the lower the rate number, the higher the effectiveness of the electrodeposition process in providing a homogeneous metal layer uniformly covering the conductive substrate. It is essential that rate numbers are equal to or lower than 2 in order to have a homogeneous metal layer which advantageously enables uniformly covering the surface of the conductive substrate.

TABLE 2

| | |
|---|---|
| 1 | Homogeneous coating with no defects (defect: not-coated area) |
| 2 | Homogeneous coating with few defects (<0.1% of not-coated |
| | area of total surface) |
| 3 | Homogeneous coating with many defects (no percolation path of |
| | not-coated surface) |
| 4 | Isles of not-coated surface area interconnected to make a |
| | percolation path |
| 5 | No coating |

In view of the above, it has been found that the composition of the invention as notably exemplified either in Example 1b or in Example 2b is successfully air and moisture stable and is thus particularly suitable for use in a process for the electrodeposition of a metal layer onto a conductive substrate, even under air atmosphere.

Also, ionic liquids suitable for use in the composition of the invention as notably exemplified either in Example 1b or in Example 2b advantageously exhibit a good electrochemical stability in a wide electrochemical window.

Moreover, the metal layer obtained by electrodeposition onto a conductive substrate according to the process of the invention is advantageously homogeneous so as to uniformly cover the surface of the conductive substrate and is also well adhered to the conductive substrate.

On the other side, the electrolyte mixture prepared according to

Comparative Example 1 or Comparative Example 2 was not suitable for use in a process for the electrodeposition of a metal layer onto a conductive substrate under air atmosphere.

1. A composition comprising:
(I) at least one ionic liquid of formula (I-a) or of formula (I-b):
[R_{F}-CFR′_{F}-SO₃]⁻ A⁺ (I-a)
[(R_{F}-CFR′_{F}-SO₂)₂N]⁻ A⁺ (I-b)
wherein:
R_{F} is a C₁-C₂₅ fluoroalkyl group, optionally comprising one or more than one catenary ethereal oxygen atoms,
R′_{F} is —F or a —CF₃ group, and
A⁺ is an organic cation selected from the group consisting of tetraalkylammonium, pyridinium, imidazolium, piperidinium, pyrrolidinium, amidinium and guanidinium groups, and
(II) at least one metal salt of formula (II):
MeₙBₘ (II)
wherein:
Me^{m+} is a metal cation derived deriving from a metal (Me) selected from the group consisting of groups IB, IIB, IVB, VB, VIB, IIIA, IVA and VIII (8, 9, 10) of the Periodic Table, wherein m is the valence of said metal cation, and
Bⁿ⁻ is an inorganic anion, wherein n is the valence of said inorganic anion.

2. The composition according to claim 1, said composition comprising:
(I) from 20% to 95% by weight, based on the total weight of the composition, of at least one ionic liquid of formula (I-a) or of formula (I-b), and
(II) from 5% to 80% by weight, based on the total weight of the composition, of at least one metal salt of formula (II).

3. The composition according to claim 1, wherein the ionic liquid has formula (I′-a) or formula (I′-b):
[R_{F1}-CF₂—SO₃]⁻ A⁺ (I′-a)
[(R_{F1}-CF₂—SO₂)₂N]⁻ A⁺ (I′-b)
wherein:
R_{F1} is selected from the group consisting of —CF₃, —CF₂H, —CFHCl, —CF₂CF₃, —CFHCF₃, —CFHOCF₃, —CF₂CF₂CF₃, —CF₂OCF₂CF₃, —CFHOCF₂CF₃, —CF₂OCFHCF₃, —CF₂OCF₂CF₂H, —CF₂OCF₂CF₂Cl, —CF₂OCFClCF₂Cl, —CFHOCF₂CF₂CF₃, —CF₂OCF₂CF₂OCF₂CF₂CF₃ and —CF₂OCF(CF₃)OCF₂CF₂CF₃, and
A⁺is an organic cation selected from the group consisting of tetraalkylammonium, pyridinium, imidazolium, piperidinium, pyrrolidinium, amidinium and guanidinium groups.

4. The composition according to claim 1, wherein the tetraalkylammonium group has formula (I-A):
[NR¹R²R³R⁴]⁺ (I-A)
wherein R¹, R², R³ and R⁴, equal to or different from each other, are independently selected from the group consisting of C₁-C₂₅ straight-chain, branched or cyclic, optionally substituted, alkanes or alkenes, and C₆-C₂₅, optionally substituted, aryl or heteroaryl groups.

5. The composition according to claim 1, wherein the pyridinium group has formula (I-B): 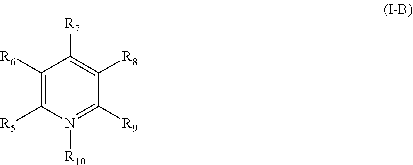
wherein R⁵, R⁶, R⁷, R⁸, R⁹ and R¹⁰, equal to or different from each other, are independently selected from the group consisting of hydrogen atoms, halogen atoms, C₁-C₂₅ straight-chain, branched or cyclic, optionally substituted, alkanes or alkenes, and C₆-C₂₅, optionally substituted, aryl or heteroaryl groups.

6. The composition according to claim 1, wherein the amidinium group has formula (I-C): 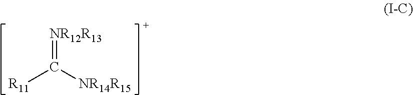
wherein R¹¹, R¹², R¹³, R¹⁴ and R¹⁵, equal to or different from each other, are independently selected from the group consisting of hydrogen atoms and C₁-C₂₅ straight-chain, branched or cyclic, optionally substituted, alkanes or alkenes, optionally comprising heteroatoms.

7. The composition according to claim 1, wherein the guanidinium group has formula (I-D): 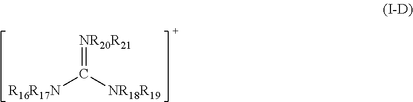
R¹⁶, R¹⁷, R¹⁸, R¹⁹, R²⁰ and R²¹, equal to or different from each other, are independently selected from the group consisting of hydrogen atoms and C₁-C₂₅ straight-chain, branched or cyclic, optionally substituted, alkanes or alkenes, optionally comprising heteroatoms.

8. The composition according to claim 1, wherein the metal salt has formula (II″):
Me″ₙB″ₘ (II″)
wherein:
Me″^{m+} is a metal cation derived from a metal (Me) selected from the group consisting of aluminium (Al), titanium (Ti), zirconium (Zr), hafnium (Hf), vanadium (V), niobium (Nb), tantalum (Ta), molybdenum (Mo) and tungsten (W), wherein m is the valence of said metal cation, and
B″ⁿ⁻ is an inorganic anion selected from the group consisting of halides, oxohalides, nitrates (—NO₃⁻), sulphates (—SO₄²⁻), sulphites (—SO₃²⁻), sulphamates (—NH₂SO₃₋), oxides (—O²⁻), hydroxides (—OH⁻), phosphates (—PO₄³⁻) and phosphites (—PO₃³⁻).

9. An electrodeposition process comprising:
(i) providing an electrolytic cell comprising:
a conductive substrate, and
a positive electrode,
said conductive substrate and said positive electrode being immersed in the composition according to claim 1; and
(ii) driving an electric current through the electrolytic cell provided in step (i).

10. The electrodeposition process according to claim 9, said process being carried out under air atmosphere.

11. The electrodeposition process according to claim 9, said process being carried out at a temperature of at most 120° C.

12. The electrodeposition process according to claim 9, wherein the conductive substrate is made of a metal selected from the group consisting of groups IB, IIB, IVB, VB, VIB, IIIA, IVA and VIII (8, 9, 10) of the Periodic Table.

13. The electrodeposition process according to claim 9, wherein the conductive substrate is made of glassy carbon.

14. A metal-coated assembly obtainable by the electrodeposition process according to claim 12, said metal-coated assembly comprising:
a conductive substrate, and
adhered to at least a portion of at least one surface of said conductive substrate, a layer made of a metal (Me) selected from the group consisting of groups IB, IIB, IVB, VB, VIB, IIIA, IVA and VIII (8, 9, 10) of the Periodic Table,
wherein said conductive substrate is made of a metal selected from the group consisting of groups IB, IIB, IVB, VB, VIB, IIIA, IVA and VIII (8, 9, 10) of the Periodic Table.

15. A metal-coated assembly obtainable by the electrodeposition process according to claim 13, said metal-coated assembly comprising:
a conductive substrate, and
adhered to at least a portion of at least one surface of said conductive substrate, a layer made of a metal (Me) selected from the group consisting of groups IB, IIB, IVB, VB, VIB, IIIA, IVA and VIII (8, 9, 10) of the Periodic Table,
wherein said conductive substrate is made of glassy carbon.

16. The composition according to claim 1, wherein -Me^{m+} is a metal cation derived from a metal (Me) selected from the group consisting of groups IVB, VB, VIB and IIIA of the Periodic Table, wherein m is the valence of said metal cation.

17. The electrodeposition process according to claim 12, wherein the conductive substrate is made of a metal selected from the group consisting of iron (Fe), copper (Cu), nickel (Ni), chromium (Cr), manganese (Mn), molybdenum (Mo), titanium (Ti), tin (Sn), zinc (Zn), palladium (Pd), platinum (Pt), silver (Ag), iridium (Ir), indium (In), lead (Pb), tungsten (W), vanadium (V), copper (Cu) and ruthenium (Ru).

18. The metal-coated assembly according to claim 14, wherein the layer is made of a metal (Me) selected from the group consisting of groups IVB, VB, VIB and IIIA of the Periodic Table.

19. The metal-coated assembly according to claim 14, wherein the conductive substrate is made of a metal selected from the group consisting of iron (Fe), copper (Cu), nickel (Ni), chromium (Cr), manganese (Mn), molybdenum (Mo), titanium (Ti), tin (Sn), zinc (Zn), palladium (Pd), platinum (Pt), silver (Ag), iridium (Ir), indium (In), lead (Pb), tungsten (W), vanadium (V), copper (Cu) and ruthenium (Ru).

20. The metal-coated assembly according to claim 15, wherein the layer is made of a metal (Me) selected from the group consisting of groups IVB, VB, VIB and IIIA of the Periodic Table.
